(19) United States
(12) Patent Application Publication
(10) Pub. No.: US 2010/0036475 A1
(43) Pub. Date: Feb. 11, 2010

(54) METHODS AND APPARATUS FOR EXTRALUMINAL FEMOROPOPLITEAL BYPASS GRAFT

(76) Inventor: Wilifrido Castaneda, New Orleans, LA (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(21) Appl. No.: 12/298,368

(22) PCT Filed: Apr. 25, 2007
(86) PCT No.: PCT/US07/67422

§ 371 Date: Jun. 2, 2009

Related U.S. Application Data
(60) Provisional application No. 60/745,854, filed on Apr. 27, 2006.

(51) Int. Cl. A61F 2/06; A61B 17/08

(52) U.S. Cl. 623/1.13; 606/153

(57) ABSTRACT

The present invention is directed to extraluminal femoropopliteal bypass grafts and methods and instruments for inserting the same. In an embodiment, the invention includes a method for inserting a femoropopliteal bypass graft including forming a first aperture in a first wall of a first artery, forming a second aperture in a second wall of the first artery, forming an extraluminal tract between the second aperture and a second artery, forming a third aperture in the second artery, and passing the femoropopliteal bypass graft through the first and second apertures, through the extraluminal tract, and into the third aperture. In some embodiments, the invention includes a femoropopliteal bypass graft having multiple layers. In some embodiment, the invention includes instruments used for percutaneously inserting a femoropopliteal bypass graft.

### FIELD OF THE INVENTION

The present invention is related to methods and apparatus for a femoropopliteal bypass graft. More specifically, the present invention is directed methods and apparatus for an extraluminal femoropopliteal bypass graft that can be percutaneously inserted.

### BACKGROUND OF THE INVENTION

Every year atherosclerosis affects the lives of millions of patients. One manifestation of atherosclerosis is peripheral vascular disease (PVD). Although less threatening to life than vascular disease of the coronary arteries, PVD is suffered by millions of individuals worldwide and is a significant cause of major disability. The most common manifestation of PVD is intermittent claudication (lameness). Traditionally, patients with PVD are managed with conservative therapy including exercise, diet and control of the risk factors such as diabetes mellitus, hypertension, obesity, smoking and hypercholesterolemia. Only about 25% of PVD patients require surgical treatment. Of the patients that require surgery, about 25% require it because of disabling intermittent claudication, while the remaining 75% need surgery because of pain, ischemic ulcers, or gangrene.

Traditional surgical treatment usually consists of the placement of a surgical bypass graft that connects the proximal segment of the blocked artery with a site distal to the block. Most commonly, the bypass graft is created with the internal saphenous vein which is resected and connected in a reversed fashion to the affected blocked artery. Sometimes the venous valves of the saphenous vein are removed and the bypass graft is created using the saphenous vein in-situ. In the absence of using the saphenous vein, a synthetic graft can be used as the bypass graft. Many synthetic grafts are made of expanded polytetrafluoroethylene (ePTFE). The term “patency” with respect to bypass graft refers to the graft remaining open and/or unobstructed. In general, long term patency is better with saphenous vein grafts than with synthetic grafts. Patency of the reversed saphenous vein femoropopliteal bypass graft varies from about 63% to about 88% after five years.

Where the saphenous vein is used for the graft, the open surgical procedure involves a surgeon making an incision in the thigh along the portion of the saphenous vein to be removed for use as the bypass graft and then dissecting and removing the vein. Once the vein is removed, the small branches of the vein are tied off. Next, an incision is made in the groin to expose the femoral artery. Another incision is made near the inside of the back of the knee to expose the popliteal artery. The femoral artery and the popliteal artery are then isolated and clamped (with vascular clamps) to block the flow of blood while the graft is being attached. The piece of the saphenous vein to be used as the graft is then tunneled along the femoral artery from the groin to the knee. One end of this vein graft is stitched into the femoral artery at the groin, and the other end of the vein graft is stitched into the popliteal artery at the knee. Once the graft is attached, blood is passed through the vein graft to check for any leaks, which, if found, are repaired. The vascular clamps are then removed, allowing blood to flow through the graft to the lower leg. This open surgical procedure requires a significant hospital stay for recovery (7-10 days) and carries with it a significant incidence of morbidity and mortality (4%-6%).

Accordingly, there is a need to for a minimally invasive method and apparatus for the insertion of an extraluminal femoropopliteal bypass graft.

### SUMMARY OF THE INVENTION

The present invention is directed to extraluminal femoropopliteal bypass grafts and methods and instruments for inserting the same. In an embodiment, the invention includes a method for percutaneous insertion of a femoropopliteal bypass graft including forming a first aperture in a first wall of a first artery, forming a second aperture in a second wall of the first artery, forming an extraluminal tract between the second aperture and a second artery, forming a third aperture in the second artery, the extraluminal tract providing fluid communication between the second aperture and the third aperture, passing the femoropopliteal bypass graft through the first and second apertures, through the extraluminal tract, and into the third aperture so that a first end of the femoropopliteal bypass graft is disposed within the first artery and a second end of the femoropopliteal bypass graft is disposed with the second artery.

In an embodiment, the invention includes a femoropopliteal bypass graft including a first layer forming a cylinder having a first end and a second end, the first layer defining a lumen, the first layer comprising a biocompatible polymer; a second layer forming a cylinder having a first end and a second end; the second layer disposed over the first layer; a third layer forming a cylinder having a first end and a second end;

the distance between the first end and the second end of the third layer being at least one centimeter less than the distance between the first end and the second end of the second layer.

The above summary of the present invention is not intended to describe each discussed embodiment of the present invention. This is the purpose of the figures and the detailed description that follows.

### DRAWINGS

The invention may be more completely understood in connection with the following drawings, in which:

FIG. 1 is a schematic view of significant arteries found in the upper leg.

FIG. 2 is an enlarged schematic view of a portion of FIG. 1.

FIG. 3 is a schematic view of the insertion of endovascular instruments into the common femoral artery.

FIG. 4 is an enlarged schematic view of a portion of FIG. 1, showing insertion of endovascular instruments into the arterial lumen.

FIG. 5 is a schematic view of a cannula disposed in an arterial lumen in a first rotational state.

FIG. 6 is a schematic view of a cannula disposed in an arterial lumen in a second rotational state.

FIG. 7 is a schematic view of endovascular instruments making an extraluminal tract though tissue surrounding the femoral and popliteal arteries.

FIG. 8 is a schematic view of endovascular instruments disposed against the wall of the superficial femoral artery.

FIG. 9 is a schematic view of a deployed femoropopliteal bypass graft in accordance with an embodiment of the invention.

FIG. 10 is a schematic view of a femoropopliteal bypass graft in accordance with an embodiment of the invention.

FIG. 11 is cross-sectional view of the femoropopliteal bypass graft of FIG. 10, taken along lines A-A′ of FIG. 10.

FIG. 12 is a schematic view of a femoropopliteal bypass graft in accordance with another embodiment of the invention.

FIG. 13 is cross-sectional view of the femoropopliteal bypass graft of FIG. 12, taken along lines B-B′ of FIG. 12.

FIG. 14 is a schematic view of a femoropopliteal bypass graft in accordance with another embodiment of the invention.

FIG. 15 is cross-sectional view of the femoropopliteal bypass graft of FIG. 14, taken along lines C-C′ of FIG. 14.

FIG. 16 is a cross-sectional view of a femoropopliteal bypass graft in accordance with another embodiment of the invention.

FIG. 17 is a cross-sectional view of a femoropopliteal bypass graft in accordance with another embodiment of the invention.

FIG. 18 is a cross-sectional view of a femoropopliteal bypass graft in accordance with another embodiment of the invention.

FIG. 19 is a schematic view of an introducer sheath in accordance with an embodiment of the invention.

FIG. 20 is a cross-sectional view of the introducer sheath of FIG. 19 taken along line D-D′ of FIG. 19.

FIG. 21 is a schematic view of a dilator catheter in accordance with an embodiment of the invention.

FIG. 22 is a cross-sectional view of the dilator catheter of FIG. 21 taken along line E-E′ of FIG. 21.

FIG. 23 is a schematic view of an internal cannula in accordance with an embodiment of the invention.

FIG. 24 is a cross-sectional view of the internal cannula of FIG. 23 taken along line F-F′ of FIG. 23.

FIG. 25 is a schematic view of a trocar in accordance with an embodiment of the invention.

FIG. 26 is a cross-sectional view of the trocar of FIG. 25 taken along line G-G′ of FIG. 25.

FIG. 27 is schematic view of a stylet in accordance with an embodiment of the invention.

FIG. 28 is schematic view of a stylet in accordance with another embodiment of the invention.

FIG. 29 is a schematic view of a stylet fitted with a trocar in a first configuration in accordance with an embodiment of the invention.

FIG. 30 is a schematic view of a stylet fitted with a trocar in a second configuration in accordance with an embodiment of the invention.

FIG. 31 is a schematic view of a double balloon catheter in accordance with an embodiment of the invention.

FIG. 32 is a cross-sectional view of the double balloon catheter of FIG. 31, taken along line I-I′ of FIG. 31.

FIG. 33 is a schematic view of a double balloon catheter in accordance with another embodiment of the invention.

FIG. 34 is a cross-sectional view of the double balloon catheter of FIG. 33, taken along line J-J′ of FIG. 33.

FIG. 35 is a cross-sectional view of the double balloon catheter of FIG. 33, taken along line K-K′ of FIG. 33.

FIG. 36 is a perspective view of the distal end of the double balloon catheter of FIG. 33.

FIG. 37 is a schematic view of a tunneling instrument in accordance with an embodiment of the invention.

FIG. 38 is a cross-sectional view of the tunneling instrument of FIG. 37 taken along line L-L′ of FIG. 37.

FIG. 39 is a schematic view of a tunneling instrument in accordance with another embodiment of the invention.

FIG. 40 is a schematic view of a guide wire in accordance with an embodiment of the invention.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Embodiments of the present invention include extraluminal femoropopliteal bypass grafts, instruments used for inserting the same, and percutaneous methods for inserting the same. While not intending to be bound by theory, the percutaneous insertion of extraluminal femoropopliteal bypass grafts is less traumatic than traditional open surgical techniques and therefore it is believed that the methods and apparatus described herein will result in a faster recovery time for patients undergoing the procedure as well as lower incidence of morbidity and mortality.

The term “percutaneous”, as used herein, shall refer to a procedure effected or performed through the skin. Thus, a procedure performed percutaneously would stand in contrast to a procedure performed through traditional open surgery.

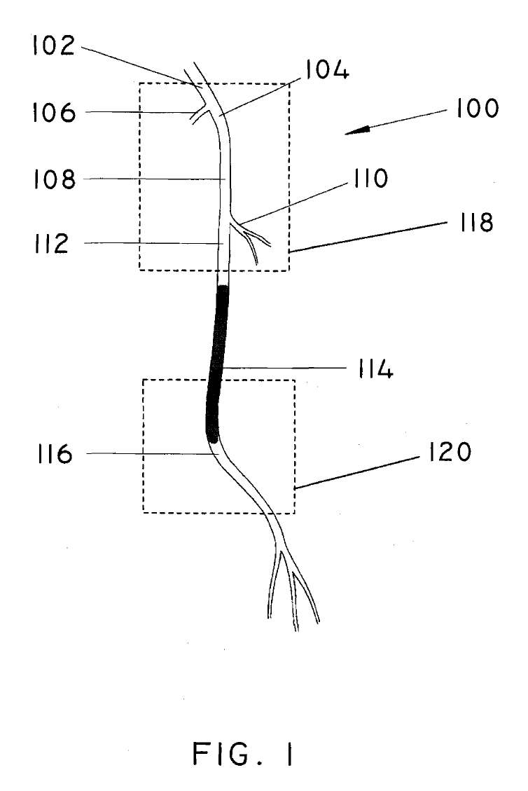

By way of reference, significant vasculature within the leg of a patient will now be described. Referring to FIG. 1, a schematic view (not to scale) is shown of significant arteries 100 found in the upper leg. The common iliac artery 102 branches into the external iliac artery 104 and the internal iliac artery 106. The external iliac artery 104 turns into the common femoral artery 108 which in turn branches into the deep femoral artery 110 and the superficial femoral artery 112. The superficial femoral artery 112 continues downward and turns into the popliteal artery 116 near the knee joint (not shown). The arteries 100 shown in FIG. 1 are depicted with an occlusion 114 (or blockage) in the area between the superficial femoral artery 112 and the popliteal artery 116. Methods and apparatus of the present invention can be used to insert a femoro-popliteal bypass graft to route blood flow around the occlusion 114.

### Insertion Methods

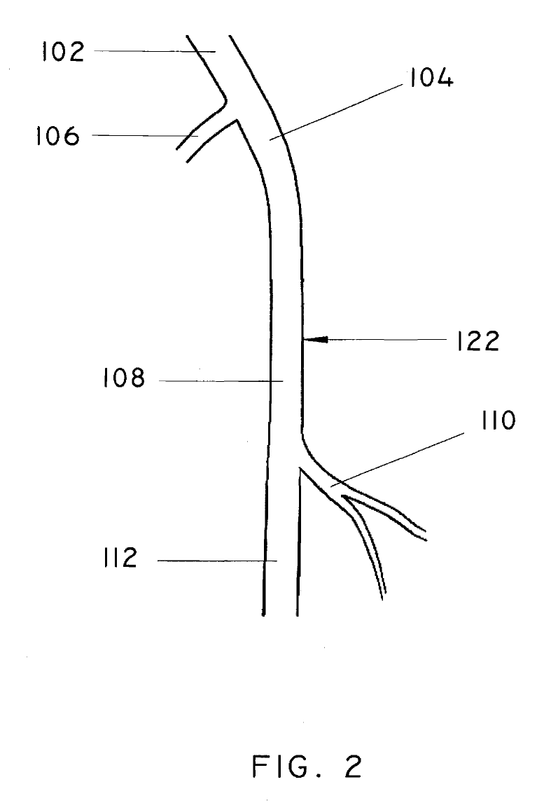

Insertion methods of the invention can include placement of an occlusive device, such as a balloon catheter, to interrupt blood flow during the insertion of a femoropopliteal bypass graft. The occlusive device can be inserted in various places in the vasculature of a patient. In an embodiment, the contralateral common femoral artery is percutaneously punctured for insertion of an occlusive device. FIG. 2 is an enlarged schematic view of a portion of the arteries shown in FIG. 1, showing one insertion point 122 for an occlusive device.

To begin, the skin site can be prepared with drapes and towels, and an anesthetic, such as 1% lidocaine, can be injected into the skin and the perivascular tissue. A small nick can be made in the skin about 1 to 2 cm beyond the intended site of arterial entry and the subcutaneous tissue gently dissected with a clamp to allow smoother entry of the apparatus. The artery can then be cannulated using either a single-wall entry or a double-wall entry technique. The needle is typically advanced at an angle of about 45 degrees to about 60 degrees with respect to the length-wise axis of the artery. After the needle is positioned within the lumen of the artery, a guide wire is passed through the lumen of the needle, and the needle is removed. The guide wire can then be advanced to the area within the arteries where the occlusive balloon catheter is to be positioned and deployed. Referring to FIG. 3, a guide wire 124 is shown passing through an insertion point 122 and traveling up the common femoral artery 108 to the external iliac artery 104. After the guide wire 124 is in the proper position, an occlusive device can then be passed over the guide wire 124 to a desired position for later deployment.

Many different types of balloon catheters can be used as an occlusive device. In some embodiments, the occlusive device is an occlusive double balloon catheter. Examples of double occlusive balloon catheters are shown in FIGS. 31-36 and described in more detail below. An occlusive double balloon catheter has two inflatable portions (balloons) that can be inflated in the same artery or in different arteries. In an embodiment, an occlusive double balloon catheter is deployed with one balloon in the internal iliac artery 106 and the other balloon in the common iliac artery 102. In an alternative embodiment, one balloon is deployed in the deep femoral artery 110 and the other balloon in the external iliac artery 104. Once deployment of the occlusive balloon catheter is achieved, the catheter shaft may be secured in place with the balloons deflated.

Next, the femoropopliteal graft is inserted into the patient. In many embodiments, the first step is establishing access to the desired artery at the sites where the graft will interface with the artery. To establish access, a first aperture can be formed in the artery using various techniques. Depending on several factors, (including the preferences of the person performing the procedure, the location of the occlusion, etc.) the first aperture may be formed in either the popliteal artery 116 or the common femoral artery 108. If the popliteal artery 116 is being accessed, the patient is placed in the prone decubitus and following surgical scrub, percutaneous puncture of the popliteal artery 116 is performed with a needle or other instrument under fluoroscopic or ultrasound guidance. Alternatively, the patient remains in the supine decubitus if the contralateral common femoral artery 108 is to be accessed percutaneously. Either of these two arteries can be percutaneously accessed using endovascular surgical techniques known to those of skill in the art.

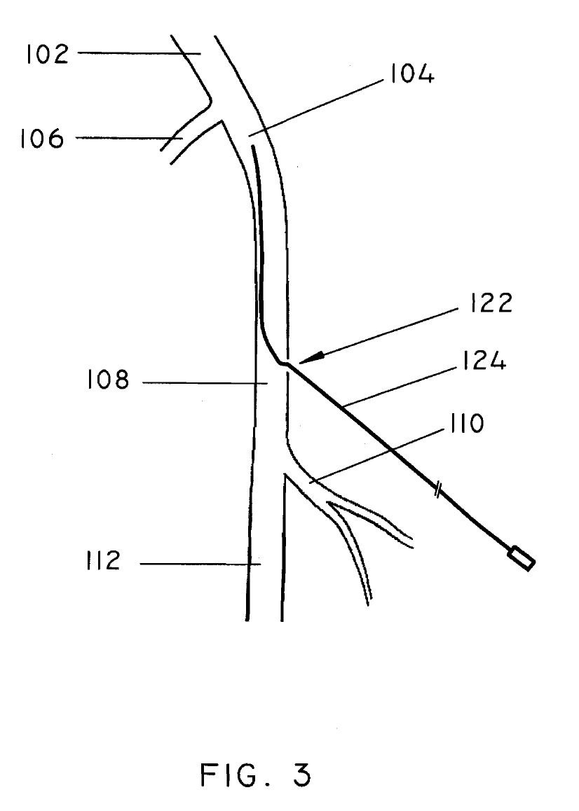

By way of example, insertion of a graft using the popliteal artery 116 as an access point is illustrated herein. FIG. 4 shows a portion 120 (see FIG. 1) of the arteries in the leg including the popliteal artery 116 and an obstruction 114. In this embodiment of the method, a needle is advanced through the artery wall 130 to form a first aperture. Once access to the artery 116 is established, a guide wire 128 is introduced into the proximal segment of the popliteal artery 116. In an embodiment, the guide wire is approximately 0.035 inches in diameter. However, guide wires with other diameters can be used. The needle is then removed and an introducer sheath (not shown) is advanced over the guide wire 128 and into the arterial lumen 132. In an embodiment, the introducer sheath is 8 Fr (or 8 mm in circumference). However, it will be appreciated that the introducer sheaths can also be either larger or smaller in circumference. Some measurements disclosed herein include reference to French sizes as used by those of skill in the medical arts. French sizes (Fr.) are defined as millimeters in circumference

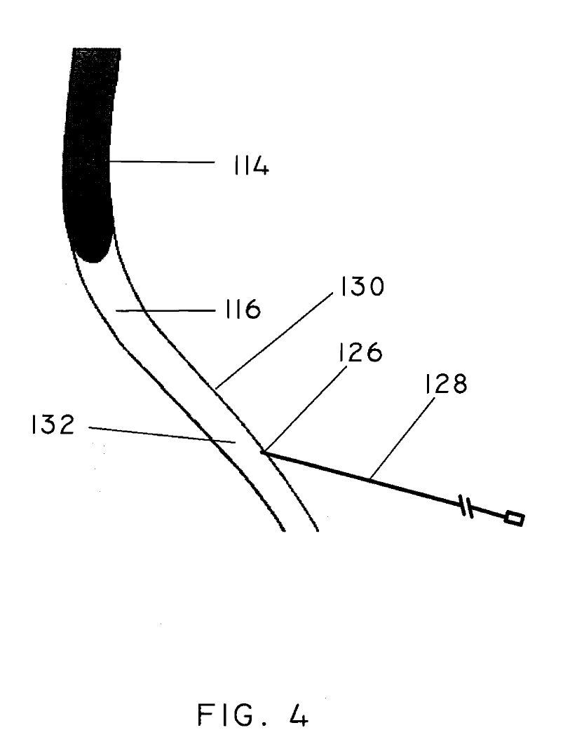

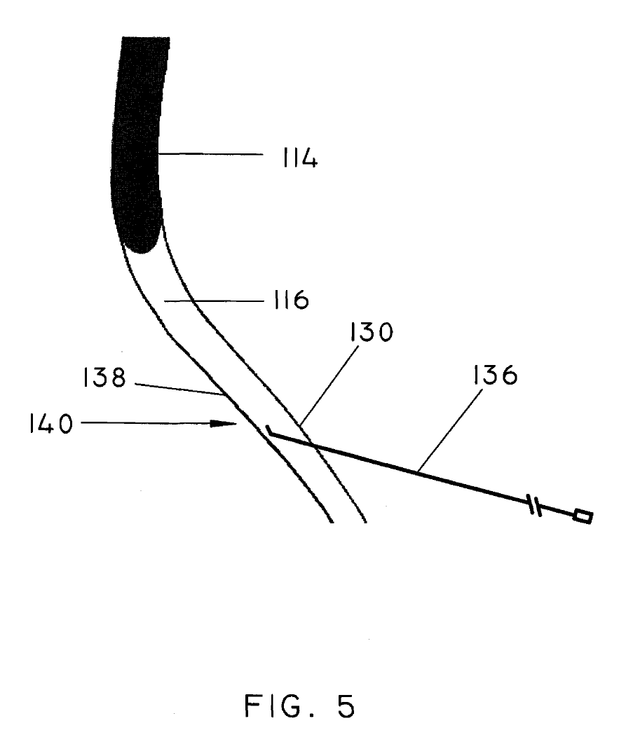

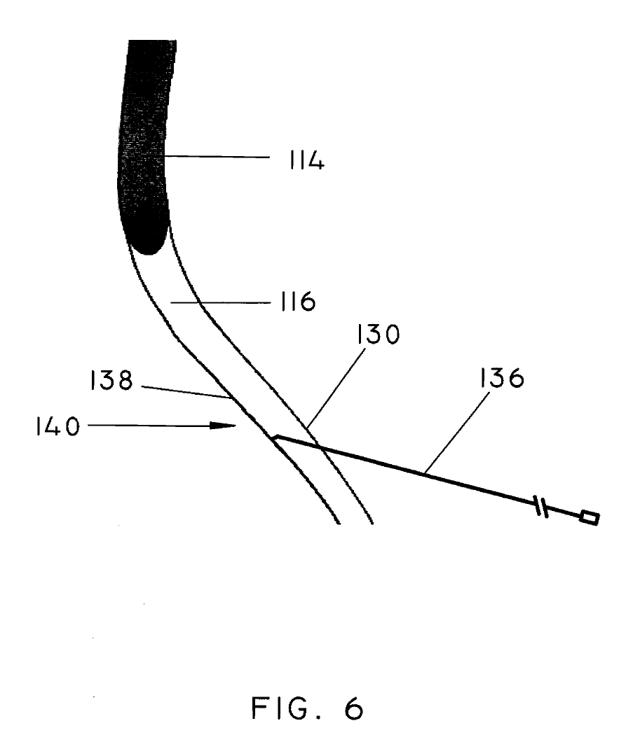

A dilator catheter/inner cannula combination are then advanced through the sheath over the guide wire 128 until the tip of the dilator catheter is within the arterial lumen. The guide wire 128 is then removed and a trocar/stylet combination is advanced through the inner cannula until they reach a position within the tip of the inner cannula. FIG. 5 shows the position of the dilator catheter/inner cannula 136 within the arterial lumen. As shown in FIG. 5, after insertion into the arterial lumen, the tip of the dilator catheter/inner cannula 136 is disposed at an angle facing inward from the main axis of the dilator catheter/inner cannula 136. The dilator catheter/cannula 136 combination is then rotated on a medial direction under fluoroscopic or preferably ultrasound guidance until it reaches a position as shown in FIG. 6. After rotation, the tip is disposed at an angle from main axis of the dilator catheter/inner cannula facing outward toward the far side of the arterial wall. A locked trocar/stylet combination is then advanced across the arterial wall 138 into the perivascular tissues 140 forming a second aperture in the artery. The second aperture can be on the opposite side of the artery from the first aperture. The distance along the length of the artery from the first aperture to the second aperture is sufficiently long so as to accommodate the end of a bypass graft. In some embodiments, this distance is greater than about 0.5 cm. In some embodiments, this distance is less than about 10 cm.

In this example, the stylet is then removed and a small amount of contrast medium is injected to verify the extravascular position of the trocar tip. Subsequently, an angled stiff hydrophilic guide wire or a precurved Nitinol guide wire (0.035 inches in diameter) is advanced through the trocar into the perivascular tissues along the popliteal artery. In some embodiments, the guide wire is 0.035 inches in diameter. However, guide wires with larger or smaller diameters can also be used. The combination of the trocar/inner cannula/dilator catheter/introducer sheath is then advanced over the guide wire into the soft tissues across the site of arterial perforation.

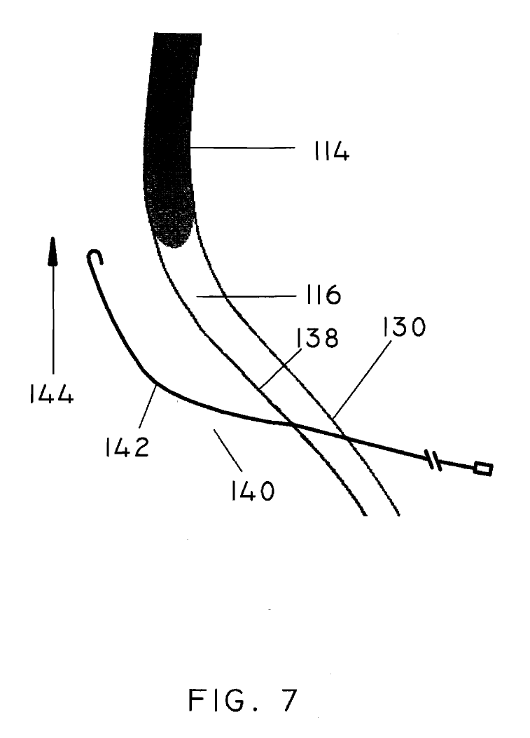

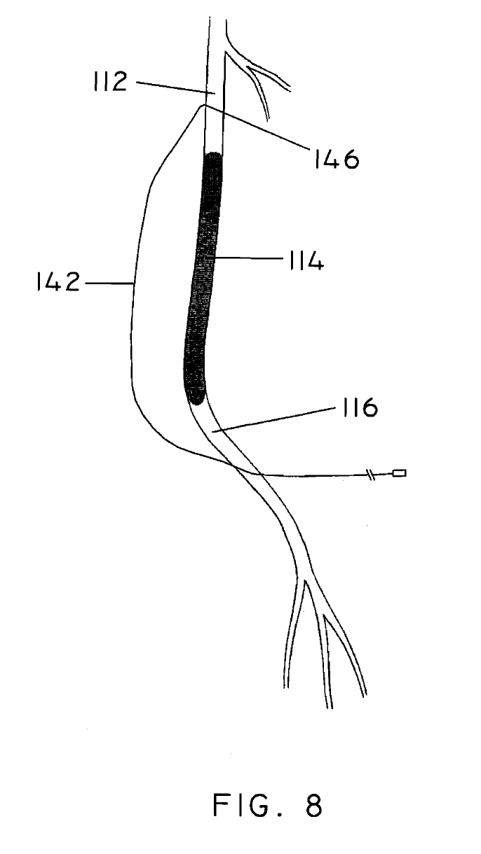

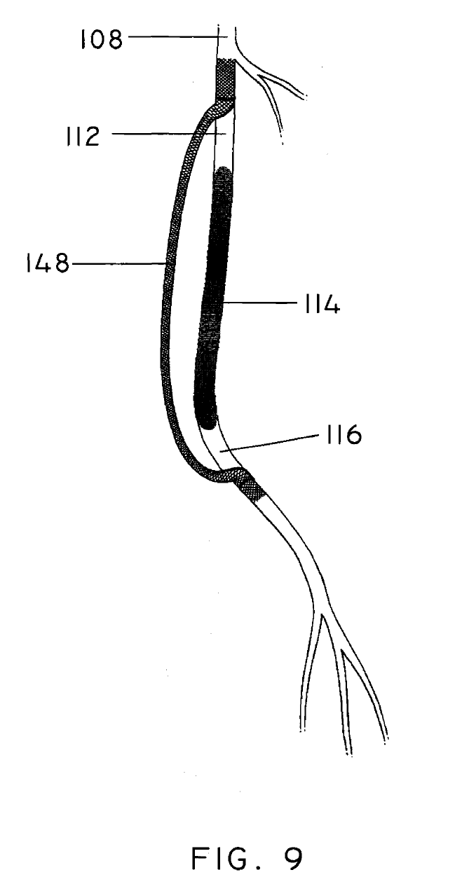

Next, blunt atraumatic dissection of an extraluminal tract is performed. Atraumatic dissection of the extraluminal tract can be performed under fluoroscopy or alternatively ultrasound, using a variety of techniques. In one approach illustrated in FIG. 7, a precurved or stiff angled guide wire 142 is advanced through the perivascular tissues 140 along the popliteal 116 and superficial femoral artery on a cephalad direction 144 with the steering help of the precurved dilator catheter (not shown) and the torque of the guide wire 142 until it reaches a desired site 146 (shown in FIG. 8) for re-entry into the vasculature, such as into the superficial femoral artery 112. Alternatively, the re-entry site could be in the common femoral artery 108 (shown in FIG. 1).

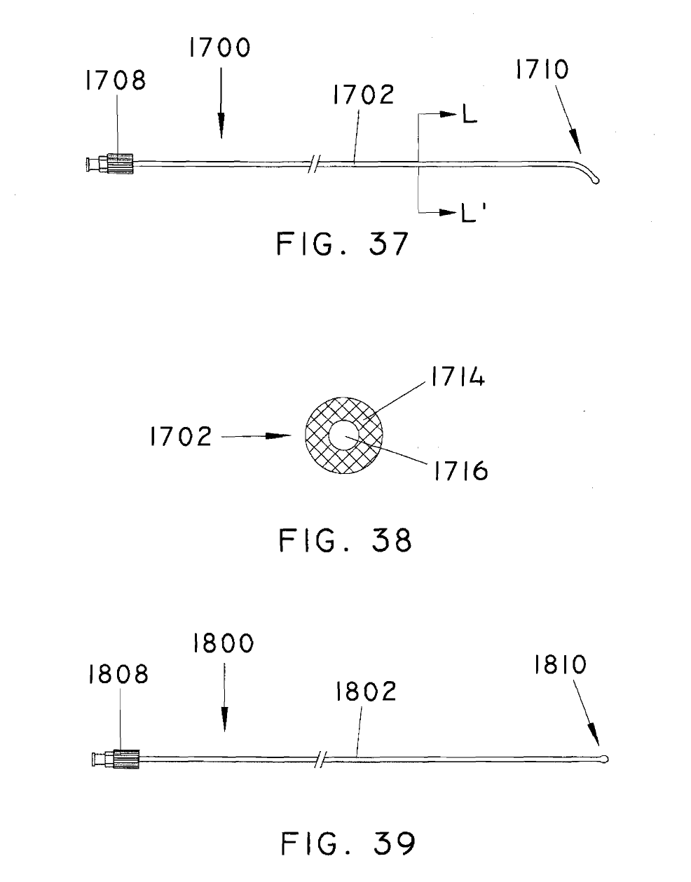

In an alternative approach to dissecting an extraluminal tract, once the introducer sheath has been advanced over the dilator catheter/inner cannula combination into the perivascular tissues, the dilator catheter/inner cannula combination can be removed and replaced by an angled or straight tunneling device (such as those shown in FIGS. 37-39), that is then used to help steer and advance the guide wire in a cephalad direction.

In yet another alternative approach to dissecting an extraluminal tract, a torque controlled angled catheter and an angled stiff hydrophilic guide wire are used to make the extraluminal tract. In this method, the guide wire is advanced into the perivascular tissues until it forms a loop, this loop is then advanced with the help of the torque controlled angled catheter.

Once the desired site 146 for re-entry into the vascular lumen has been reached, keeping the guide wire and introducer sheath in place, all other devices are removed and are replaced for the dilator catheter/inner cannula/trocar combination (not shown), which are then advanced together with the introducer sheath to the selected proximal vascular re-entry site. At this time, the occlusive device (such as an occlusive balloon catheter) is activated to occlude blood flow.

The wire is then replaced by the stylet and the dilator catheter/inner cannula combination are rotated so that the angled portion of the inner cannula is facing towards the desired reentry site (the proximal superficial femoral artery or towards the common femoral artery in cases of high occlusion of the superficial femoral artery) as shown in FIG. 8. Once the position of the tip of the dilator catheter/cannula/trocar combination against the arterial wall is proven by fluoroscopy or ultrasound the stylet/trocar combination is used to perforate the arterial wall to form a third aperture.

The trocar is then removed and contrast medium is injected after aspirating blood. A guide wire is then advanced into the common iliac artery and the introducer sheath/dilator catheter/cannula/trocar combination are advanced together into the arterial lumen. While keeping the introducer sheath in place and maintaining blockage of blood flow with the occlusion device, the dilator catheter/cannula and trocar are removed and a graft (endoprosthesis) of adequate length is advanced through the introducer sheath until the proximal end is seen within the vascular lumen of the proximal superficial femoral artery or common femoral artery. Specifically, a femoropopliteal bypass graft is passed through the first and second apertures, through the extraluminal tract, and in the third aperture so that the proximal end of the graft is disposed within the vascular lumen of the superficial femoral artery or common femoral artery and the distal end of the graft is disposed within the vascular lumen of the popliteal artery.

While pulling back on the introducer sheath, the graft is released throughout the length of the extraluminal tract. In some embodiments, the graft is self-expanding and a seal is formed between the end portions of the graft and the arterial wall after the introducer sheath is withdrawn due to outward pressure on the arterial wall generated by the graft itself In other embodiments, the graft is balloon expandable and a seal is formed between the end portions of the graft and the arterial wall due to balloon expansion of the end portions of the graft. In some embodiments, the force of the graft against the wall of the arteries is sufficient to hold the graft in place such that sutures are not necessary.

A balloon catheter can be advanced over the guide wire to ensure that all portions of the graft are fully expanded. The occlusive device on balloons can then be deflated and a control angiogram can be performed to assess patency of the bypass and to check for presence or absence of leaks. In some embodiments, a vascular sealant device can then be used to close the percutaneous access site (first aperture) in the popliteal artery.

It will be appreciated that this description of a method for inserting a femoropopliteal bypass graft is provided by way of example only and it will be appreciated that certain steps in the procedure described can be performed in a different order than as provided without deviating from the spirit and scope of the invention. Embodiments of grafts that can be used in the bypass procedure will now be described in greater detail.

### Bypass Grafts

Referring now to FIG. 10, a synthetic graft 200 in accordance with an embodiment of the invention is shown. The graft has an inner layer 210. The inner layer 210 contacts materials that pass through the graft when it is deployed in the body of a patient. In an embodiment, the inner layer can be made of expanded polytetrafluoroethylene (ePTFE). ePTFE has desirable in vivo properties including biocompatibility and very little thrombogenicity. It will be appreciated that the inner layer can also be made of other biocompatible materials. By way of example, the inner layer can be made of polyethylene, polyurethane, silicone, DACRON®, and the like. In some embodiments, the inner layer 210 comprises a woven material (such as a braid). In other embodiments, the inner layer 210 comprises a non-woven material.

The inner layer 210 may optionally be impregnated with one or more active agents that prevent stenosis and thrombosis of the graft. Flow detectors may also be attached to the inner layer or embedded within the inner layer so as to be able to detect flow of a fluid through the graft 200. Such flow detectors can provide a signal that can be detected by a diagnostic apparatus (not shown) to assist in non-invasive monitoring and trouble-shooting of the graft.

A middle layer 208 is disposed over the inner layer 210. The middle layer 208 can be fastened to the inner layer 210. By way of example, the middle layer 208 can be attached to the inner layer 210 with a biocompatible adhesive or with stitches. The middle layer 208 can also be held to the inner layer 210 through a pressure-type fit. In some embodiments, the middle layer 208 can be made of a mesh material, such as a wire mesh. In some embodiments, the middle layer 208 is a tubular braid. In some embodiments, the middle layer 208 can include multiple strands of material running parallel to each other in a helical pattern. In other embodiments, a single strand of a material is used to make a tubular braid. The pitch of the strands of the material is defined as the angle between the turns of the wire and the axis of the braid. The pick is the number of turns per unit length. A higher pitch and pick will produce a tighter mesh. Conversely, a lower pitch and pick will produce a looser mesh. In an embodiment, the middle layer is a tubular braid having a diameter of about 4 to about 5 mm. In an embodiment, the middle layer has a pitch of between about 60 and 70 degrees and a pick of about 50-70 per linear inch. However, other embodiments can include other pitches and picks.

In one approach to forming a middle layer 208 from a metal mesh, the tubular braid is cut to the right length starting with a longer piece. Where a tubular braid is used that is made from multiple strands, clamps may be used to prevent the braid from unraveling or alternatively the strands are welded together. Laser-welding is one technique for welding strands together.

In some embodiments, the middle layer 208 is made starting from a Nitinol tube that is then cut using laser techniques to result in a tubular shape that has the appearance of a mesh. In some embodiments, the middle layer 208 can include strands of a shape-memory metal braided together with strands of a polymer such as ePTFE.

In a particular embodiment, the middle layer 208 is made of a shape-memory metal. Exemplary shape-memory metals are described in more detail below. In a specific embodiment, the middle layer 208 is made of Nitinol.

The middle layer 208 can provide structural integrity to the graft such that the lumen of the graft is held open when the graft is deployed in the body of a patient. In addition, the middle layer 208 can provide structural rigidity at the proximal end 204 and the distal end 202 of the graft 200 so that the ends (204, 202) can sealingly engage the walls of the artery into which they are placed. In some embodiments, grafts described herein are self-expandable and the middle layer 208 provides an outward force that causes the graft to expand radially to reach a larger diameter than the diameter when it is being moved into place. In other embodiments, grafts described here are balloon-expandable.

An outer layer 206 is disposed over the middle layer 208. The outer layer 206 can be fastened to the middle layer 208. By way of example, the outer layer 206 can be attached to the middle layer 208 with a biocompatible adhesive or with stitches. The outer layer 206 can also be held to the middle layer 208 through a pressure-type fit. The outer layer can be made of biocompatible materials. By way of example, the outer layer can be made of polyethylene, polyurethane, silicone, DACRON®, and the like. In a particular embodiment, the outer layer is made of ePTFE. In some embodiments, the outer layer 206 can include strands of a shape-memory metal braided together with strands of a polymer such as ePTFE.

FIG. 11 shows a cross-sectional view of the graft 200 of FIG. 10 taken along line A-A′. In this embodiment, the inner layer 210 and the middle layer 208 are of the same overall length. However, the outer layer 206 is shorter than the middle layer 208 such that a portion of the middle layer 208 is exposed at both the proximal end 204 and the distal end 202 of the graft 200. In some embodiments, about 1 to about 2 centimeters of the middle layer 208 is exposed at the proximal end 204, the distal end 202, or both the proximal end 204 and the distal end 202. While not intending to be bound by theory, it is believed that leaving a portion of the middle layer 208 uncovered by the outer layer 206 can aid in the process of firmly anchoring the proximal end 204 and the distal end 202 of the graft 200 into the arterial wall tissues.

In the embodiment shown, the inner layer 210, middle layer 208, and outer layer 206, have a distal end diameter 212 that is smaller than the proximal end diameter 214. While not intending to be bound by theory, it is believed that this tapered configuration allows the graft to fit in place better. This is because the arteries the graft 200 is placed into generally taper as they pass farther into the extremities (e.g., the popliteal artery generally has a smaller lumen diameter than the superficial femoral artery or the common femoral artery). However, it will be appreciated that in other embodiments, the graft can have substantially the same diameter from its proximal end 204 to its distal end 202.

Referring now to FIG. 12, another embodiment of a graft 300 is shown. In this embodiment, a structural layer 308 covers an inner layer 310. The structural layer 308 can be made of a mesh material, such as a wire mesh. In an embodiment, the structural layer 308 is made of a shape-memory metal. In an embodiment, the structural layer 308 is made of Nitinol. The structural layer 308 can provide structural integrity to the graft such that the lumen of the graft is held open. The inner layer 310 contacts materials that pass through the graft once it is deployed in vivo. The inner layer 310 can be made of biocompatible materials. By way of example, the inner layer 310 can be made of polyethylene, polyurethane, silicone, DACRON®, and the like. In a particular embodiment, the inner layer 310 is made of ePTFE. FIG. 13 is a cross-sectional view of the graft 300 of FIG. 12, taken along line B-B′.

Referring now to FIG. 14, another embodiment of a graft 400 is shown. In this embodiment, a structural layer 408 is partially covered by an outer layer 406. The structural layer 408 can be made of a mesh material, such as a wire mesh. In an embodiment, the structural layer 408 is made of a shape-memory metal. In an embodiment, the structural layer 408 is made of Nitinol. The structural layer 408 can provide structural integrity to the graft such that the lumen of the graft is held open.

The outer layer 406 can be made of biocompatible materials. By way of example, the outer layer 406 can be made of polyethylene, polyurethane, silicone, DACRON®, and the like. In a particular embodiment, the outer layer 406 can be made of ePTFE. In this embodiment, the outer layer 406 is shorter than the structural layer 408.

A wire winding 416 is disposed over the outer layer 406. In some embodiments, the wire winding 416 can comprise one or more strands of a metal wire wrapped around the outer layer 406 one or more times. In a particular embodiment, the wire winding 416 comprises two strands of Nitinol. FIG. 15 is a cross-sectional view of the graft 400 shown in FIG. 14, taken along line C-C′.

In a further embodiment not shown, the structural layer does not extend across the whole length of the graft. Instead, the structural layer is divided into two cylindrical segments that can be referred to as fixation elements. The fixation elements are typically positioned at the proximal and distal ends of the graft. The fixation elements can include a thermoelastic material. The fixation elements can include a shape-memory metal. In some embodiments, the fixation elements are woven into another layer of the graft. In other embodiments, the fixation elements are inside the lumen of graft or on the outside of the graft. The fixation elements can be positioned such that they extend beyond the ends of the other layers of the graft. By way of example, one of the fixation elements may extend from about 1 to about 2 centimeters beyond the proximal end of the graft and the other fixation element may extend from about 1 to about 2 centimeters beyond the distal end of the graft.

FIG. 16 shows a cross-sectional view of another embodiment of a graft 500 in accordance with an embodiment of the invention. A structural layer 508 is covered by an outer layer 506. A wire winding 516 is disposed over the outer layer 506. In this embodiment, the structural layer 508 is the same length as the outer layer 506.

FIG. 17 shows a cross-sectional view of another embodiment of a graft 600 in accordance with an embodiment of the invention. A structural layer 608 is disposed over an inner layer 610. The structural layer 608 is longer than the inner layer 610 such that there are portions 618 of the structural layer 608 extending beyond the inner layer 610 on both ends.

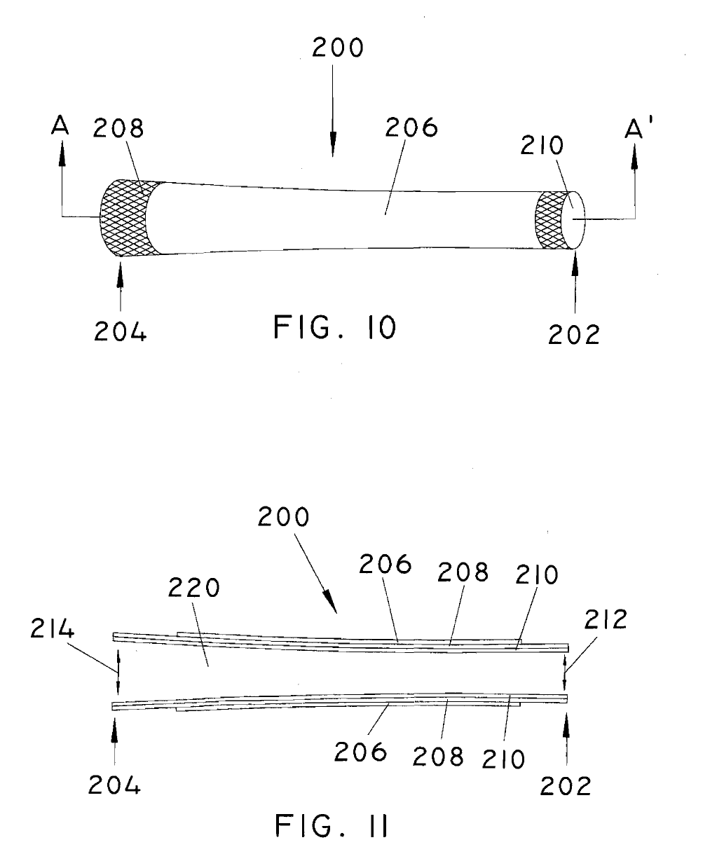

FIG. 18 shows a cross-sectional view of another embodiment of a graft 700 in accordance with an embodiment of the invention. A structural layer 708 covers an inner layer 710. An outer layer 706 covers a portion of the structural layer 708. In this embodiment, the structural layer 708 is longer than both the outer layer 706 and the inner layer 710. The inner layer 710 is longer than the outer layer 706.

### Graft Insertion Instruments

Referring now to FIG. 19, an introducer sheath 800 is shown. The introducer sheath 800 has a shaft 802. The introducer sheath 800 has a hemostasis valve assembly 810 connected to the proximal end of the shaft 802. The hemostasis valve assembly 810 may be either detachably or non-detachably connected to the shaft 802. The hemostasis valve assembly 810 acts as a seal to prevent entry of air into the circulation as well as blood or fluid loss when equipment is removed from within the sheath. In this embodiment, the hemostasis valve assembly 810 has a side port 806. The side port 806 can be used for flushing purposes. A stopcock assembly 812 is in fluid communication with the side port 806. The sheath 800 may optionally have a radiopaque marker 804 located near the distal end of the shaft 802. The radiopaque marker 804 facilitates visualization of the position of the sheath.

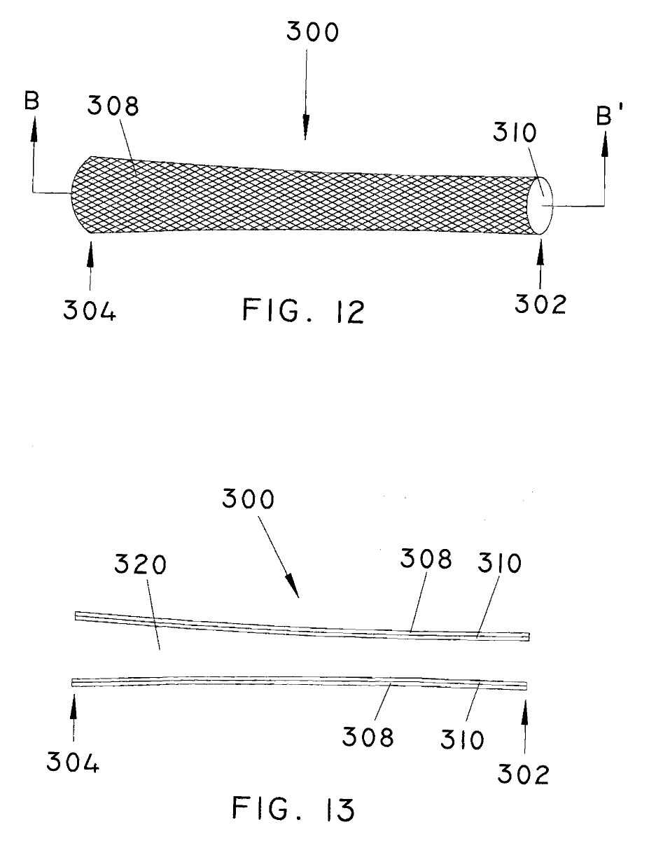

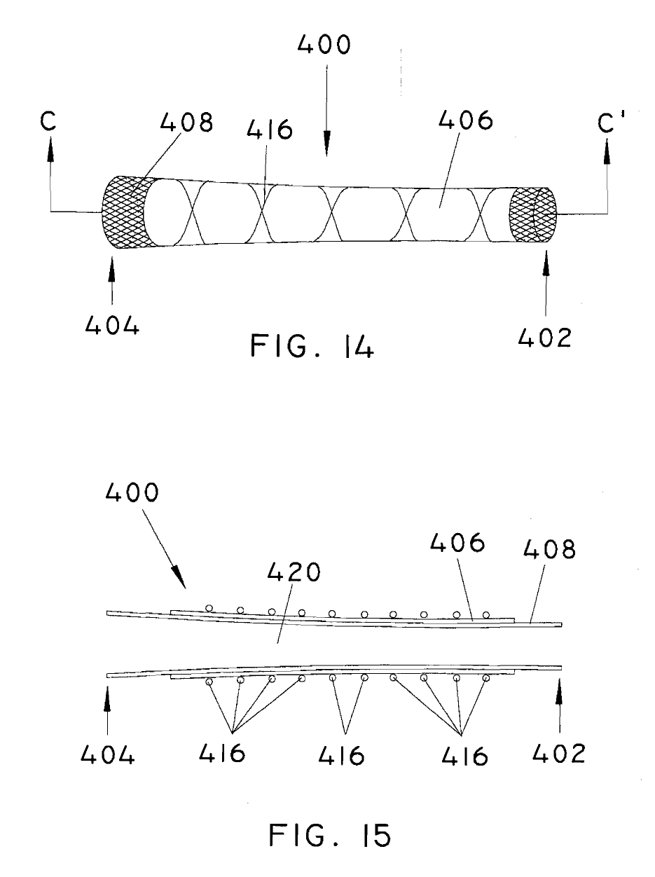

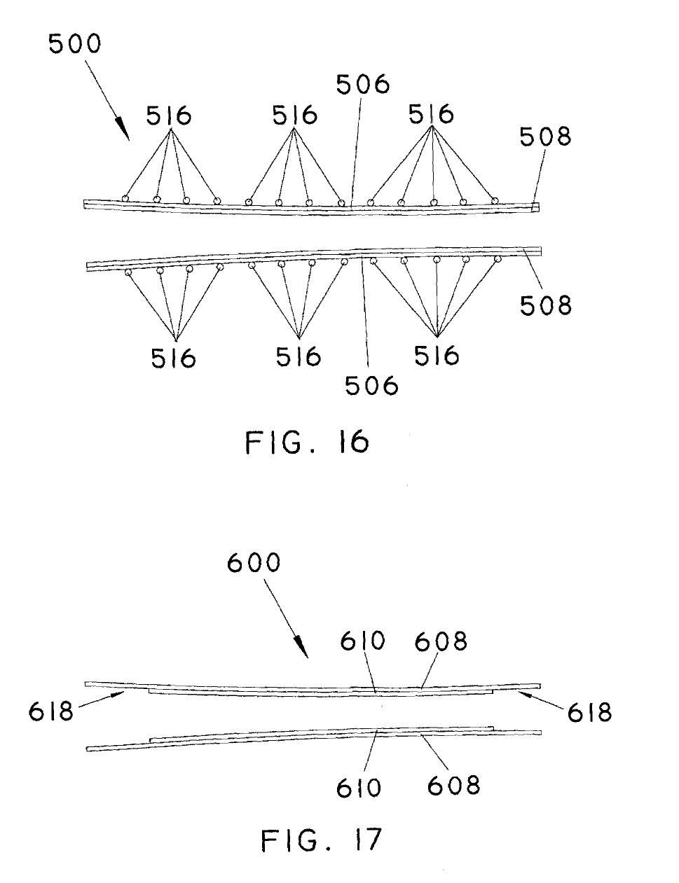

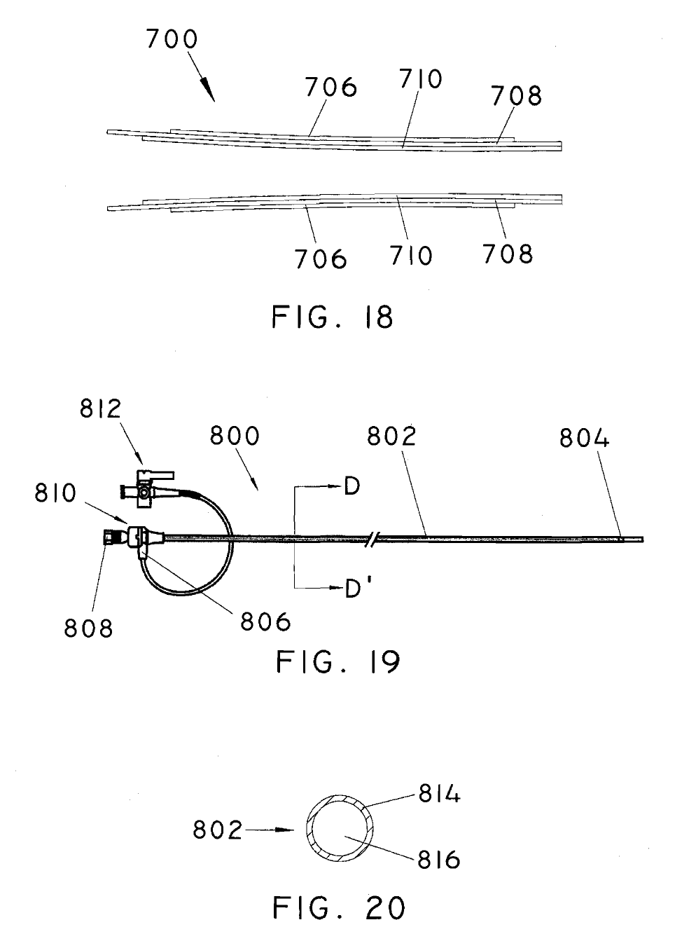

FIG. 20 is a cross-sectional view of the shaft 802 of sheath 800 taken along line D-D′ of FIG. 19. The shaft 802 has a sidewall 814 and a lumen 816. In some embodiments, the thickness of the sidewall 814 is less than or equal to 1 mm. The shaft 802 of the introducer sheath 800 can be made of many materials. In an embodiment, the shaft 802 is an extrusion of a polymer. Polymers used can include any type suitable for use with a medical device include polyurethane, polyethylene, silicone, etc. The material of the sidewall 814 can be treated to increase its ability to advance through perivascular tissues, such as by increasing its lubricity, decreasing its frictional coefficient, or enhancing the degree of hydrophilicity. The shaft 802 is constructed so as to be resistant to kinking. In an embodiment, the shaft 802 of the introducer sheath is 8 Fr (or 8 mm in circumference). However, in other embodiments, the shaft 802 is either larger or smaller in circumference.

FIG. 21 shows a dilator catheter 900. The dilator catheter 900 has a shaft 902 and a lock fitting 908 at its proximal end. By way of example the lock fitting 908 can be a LUER-LOK® type fitting or other fastener. In this embodiment, the distal end 918 of the shaft 902 is angled with respect to the central axis of the shaft 902. In an embodiment, the distal end 918 of the shaft 902 is angled by about 20 to about 30 degrees with respect to the central axis of the shaft. In an embodiment, the angled portion of the shaft is about 1 to about 2 centimeters in length.

FIG. 22 shows a cross-sectional view of the dilator catheter of FIG. 21 taken along line E-E′ of FIG. 21. The catheter has a side wall 914 defining an internal lumen 916. The side wall 914 may comprise an extrusion of a suitable polymer. In an embodiment, the side wall 914 is made of a polymer that is kinking resistant, has a low friction coefficient, and good torque control. By way of example, the side wall can be made of polyurethane, polyethylene, silicone, polytetrafluoroethylene, and the like. In some embodiments, the internal lumen 916 gradually tapers at its distal end 918 to the diameter of an internal cannula.

FIG. 23 shows an internal cannula 1000. The internal cannula 1000 has a shaft 1002 and a lock fitting 1008 at its proximal end. By way of example the lock fitting 1008 can be a LUER-LOK® type mechanism or other fastener. In an embodiment, the lock fitting 1008 of the internal cannula 1000 can engage the lock fitting (908 in FIG. 21) on a dilator catheter. In some embodiments, the internal cannula 1000 can be about 1 to 2 centimeters shorter than the dilator catheter it is designed to engage. In this example, the distal end 1018 of the shaft 1002 is angled with respect to the central axis of the shaft 1002. In an embodiment, the distal end 1018 of the shaft 1002 is angled by about 20 to about 30 degrees with respect to the central axis of the shaft. In an embodiment, the angled portion of the shaft 1002 is about 1 to about 2 centimeters in length.

FIG. 24 shows a cross-sectional view of the internal cannula 1000 of FIG. 23 taken along line F-F′ of FIG. 23. In an embodiment, the internal cannula 1000 has an outside diameter of about 0.065 inches (1.65 mm). However, in other embodiments, the internal cannula 1000 can have a larger or smaller diameter. The internal cannula 1000 has a side wall 1014 defining an internal lumen 1016. In an embodiment, the internal lumen 1016 is large enough to allow passage of a 0.053 inch trocar (1.35 mm). The side wall 1014 may comprise a metal such as stainless steel. In some embodiments, the side wall 1014 comprises a shape memory metal.

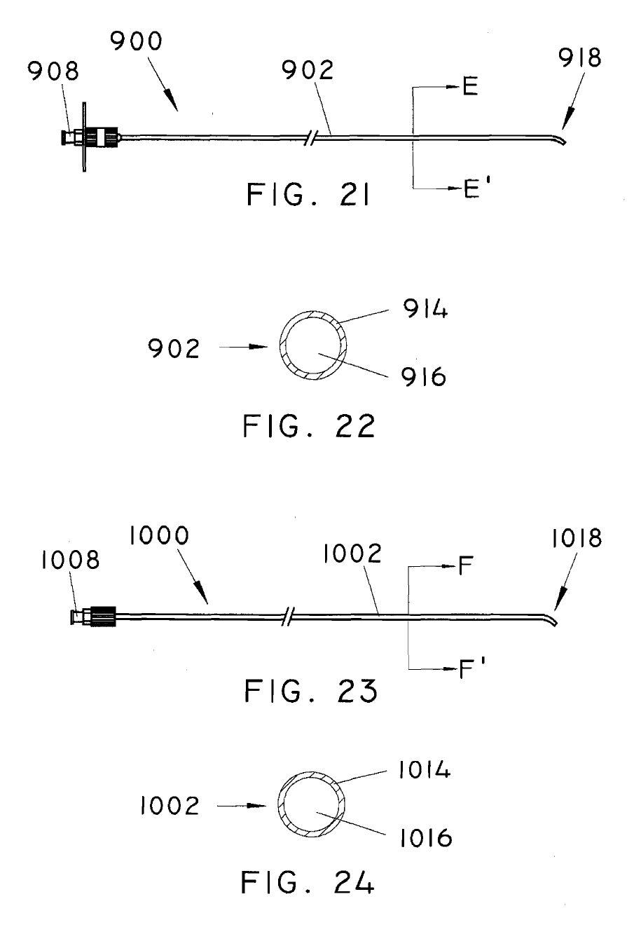

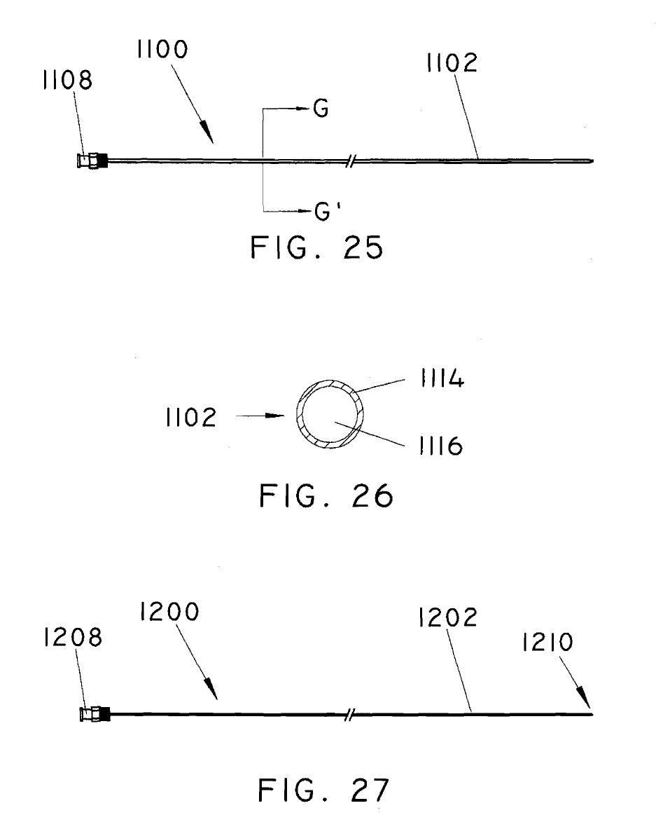

FIG. 25 shows a trocar 1100. The trocar 1100 has a shaft 1102 and a lock fitting 1108 at its proximal end. By way of example, the lock fitting 1108 can be a LUER-LOK® type mechanism or other fastener. In an embodiment, the lock fitting of 1108 can engage the lock fitting on a stylet. FIG. 26 shows a cross-sectional view of the trocar 1100 of FIG. 25 taken along line G-G′ of FIG. 25. The trocar has a side wall 1114 defining an internal lumen 1116. In some embodiments, the internal lumen is large enough to accommodate a stylet having an outer diameter of 0.035 inches (1 mm). The trocar is configured to fit within the lumen of an internal cannula. In some embodiments, the outside diameter of the trocar is about 0.053 inches (1.53 mm). However, in other embodiments, the outside diameter of the trocar is either larger or smaller than 0.053 inches. In some embodiments, the trocar 1100 is about 5 centimeters longer than an internal cannula that it is adapted to fit within. The trocar can be made of various materials including polymer, metals, alloys, etc.

FIG. 27 shows a stylet 1200. The stylet 1200 has a shaft 1202 and a lock fitting 1208 at its proximal end. In an embodiment, the stylet 1200 has an outer diameter of about 0.035 inches (1 mm). However, in other embodiments, the outer diameter is either larger or smaller than 0.035 inches (1 mm). The tip of the distal end 1210 of the stylet 1200 is sufficiently sharp to pierce an arterial wall. By way of example, the lock fitting 1208 can be a LUER-LOK® type mechanism or other fastener. In an embodiment, the lock fitting 1208 can engage the lock fitting on a trocar. The stylet 1200 is configured to fit within the lumen of a trocar. In some embodiments, the stylet 1200 is about the same length as a trocar that it is adapted to fit within.

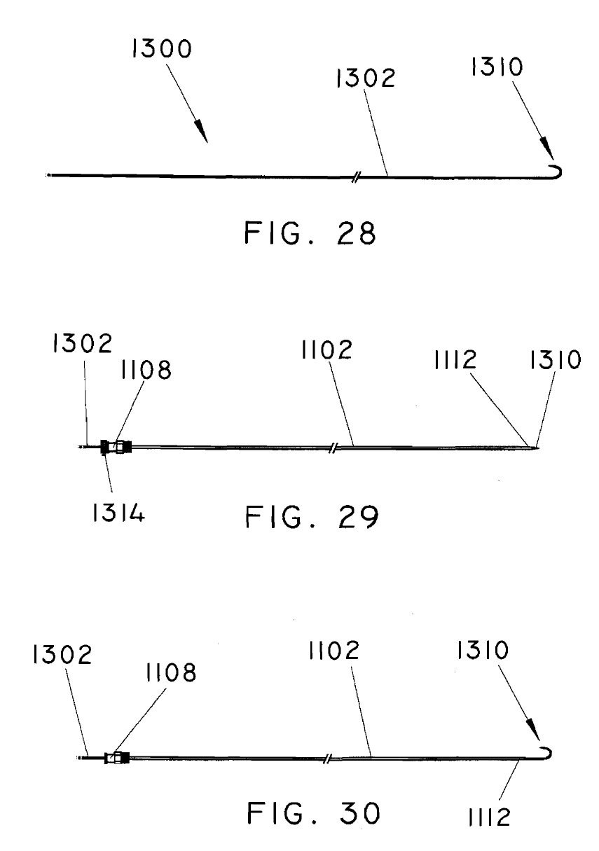

FIG. 28 shows a different embodiment of a stylet 1300. In this embodiment, the stylet 1300 has a shaft 1302 and a sharp tip 1310 that assumes a J-shape. The stylet 1300 can be made of a shape-memory metal. The stylet 1300 can be flexible such that the tip 1310 is straight when confined inside the lumen of a trocar. However, when the stylet 1300 is advanced out of the tip of the trocar, the tip 1310 reassumes the J-shaped configuration. By way of illustration, referring now to FIG. 29, the shaft 1302 of a stylet 1300 is shown being held straight within the lumen of a trocar shaft 1102. The tip 1310 of the stylet 1300 is at a position roughly 2 mm past the tip 1112 of the trocar 1100. A removable locking mechanism 1314 prevents the tip 1310 from advancing farther beyond the trocar tip 1112. In this configuration, the trocar 1100/stylet 1300 combination can be used to pierce the arterial wall because the tip 1310 is being held straight by the trocar shaft 1102. However, referring now to FIG. 30, when the removable locking mechanism 1314 is removed, the stylet tip 1310 can be advanced farther beyond the trocar tip 1112 and reassumes its J-shape. When the stylet tip 1310 assumes a J-shape it can then be safely advanced into places, such as within the arterial lumen, without the risk of inadvertently piercing other tissues.

Referring now to FIG. 31, an example of an occlusive double balloon catheter 1500 is shown. The occlusive double balloon catheter 1500 has a shaft 1502, a first balloon 1506, a first balloon inflation port 1514, a second balloon 1508, a second balloon inflation port 1512, a guide wire entry port 1510, and one or more contrast media ports 1504. In this view, the first balloon 1506 and the second balloon 1508 are shown in an inflated configuration. When positioned at a desired point within the vasculature of a patient, the balloons can be inflated to occlude blood flow.

FIG. 32 shows a cross-sectional view of the shaft 1502 of the occlusive double balloon catheter 1500 taken along line I-I′ of FIG. 31. The shaft 1502 includes a guide wire lumen 1516 for passage of a guide wire. In some embodiments, the guide wire lumen 1516 is large enough to accommodate a 0.014 inch (0.4 mm) guide wire. The guide wire lumen 1516 is in fluid communication with the contrast media ports 1504. The shaft 1502 also includes a first balloon inflation lumen 1518 and a second balloon inflation lumen 1520. The first balloon inflation lumen 1518 provides fluid communication between the first balloon inflation port 1514 and the first balloon 1506. Similarly, the second balloon inflation lumen 1520 provides fluid communication between the second balloon inflation port 1512 and the second balloon 1508.

The shaft 1502 of the double balloon catheter 1500 can be made of an extruded polymer. By way of example, the shaft 1502 of the double balloon catheter 1500 can be made of an extrusion of polyurethane, polyethylene, silicone, polytetrafluoroethylene, or the like. In an embodiment, the shaft 1502 is made of a material that is kink resistant and has a low friction coefficient.

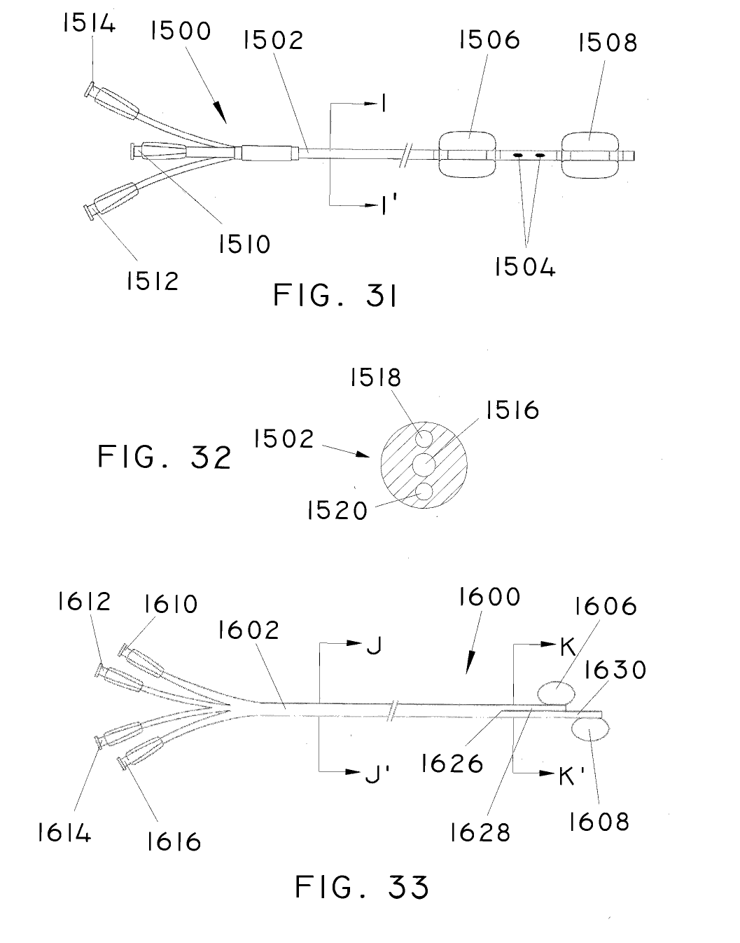

Referring now to FIG. 33, an alternative embodiment of an occlusive double balloon catheter 1600 is shown. The occlusive double balloon catheter 1600 has a shaft 1602, a first balloon 1606, a first balloon inflation port 1610, a second balloon 1608, a second balloon inflation port 1616, a first guide wire insertion port 1612 and a second guide wire insertion port 1614. In this view, the first balloon 1606 and the second balloon 1608 are shown in an inflated configuration. When positioned at a desired point within the vasculature of a patient, the balloons can be inflated to occlude blood flow. The shaft 1602 splits at a point 1626 into two distal portions 1628, 1630. In some embodiments, the point 1626 at with the shaft 1602 splits is about 20 centimeters from the distal end of the balloon catheter. One distal portion 1630 can be about 5 to about 10 centimeters longer than the other distal portion 1628.

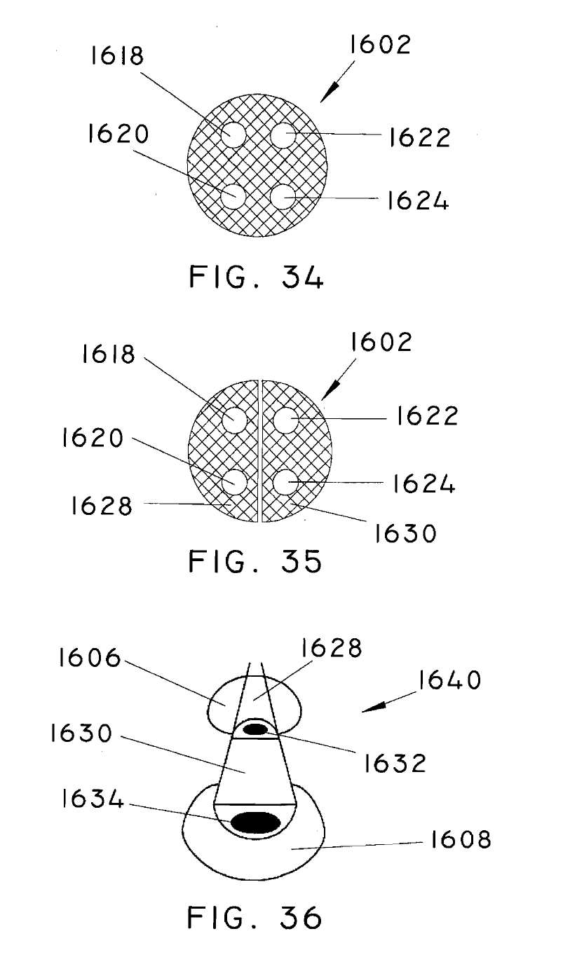

FIG. 34 shows a cross-sectional view of the shaft 1602 of the occlusive double balloon catheter 1600 taken along line J-J′ shown in FIG. 33. The shaft 1602 includes a first balloon inflation lumen 1618 and a second balloon inflation lumen 1622. The shaft 1602 also includes a first guide wire lumen 1620 and a second guide wire lumen 1624. In some embodiments, the guide wire lumens are large enough to accommodate an 0.018 inch (0.46 mm) guide wire. The first balloon inflation lumen 1618 provides fluid communication between the first balloon inflation port 1610 and the first balloon 1606. Similarly, the second balloon inflation lumen 1622 provides fluid communication between the second balloon inflation port 1616 and the second balloon 1608. In some embodiments, the occlusive double balloon catheter 1600 is introduced through a peel away introducer sheath.

FIG. 35 shows a cross-sectional view of the shaft 1602 of occlusive double balloon catheter 1600 taken along line K-K′ shown in FIG. 33. This view shows a separation between the two distal portions 1628, 1630. The two distal portions 1628, 1630 can move independently and therefore be independently positioned within the vasculature of a patient. Thus, for example, one distal portion 1628 can be positioned within one artery while the other distal portion 1630 can be positioned within another artery. FIG. 36 shows a perspective view of the distal end of the catheter. A guide wire exit port 1632 is disposed on the end of distal portion 1628. Similarly, a second guide wire exit port 1634 is disposed on the end of distal portion 1630.

The shaft 1602 of the double balloon catheter 1600 can be made of an extrusion of a polymer. By way of example, the shaft 1602 of the double balloon catheter 1600 can be made of an extrusion of polyurethane, polyethylene, silicone, polytetrafluoroethylene, or the like. In an embodiment, the shaft 1602 is made of a material that is kink resistant and has a low friction coefficient.

Referring now to FIG. 37, a tunneling instrument 1700 is shown. The tunneling instrument 1700 can be used to create a tunnel (extraluminal tract) between the proximal and distal vascular sites for insertion of the bypass graft. The tunneling instrument 1700 has a shaft 1702 and a distal end 1710. The distal end 1710 has a blunt tip to allow for the atraumatic dissection of an extraluminal tract. A portion (angled portion) of the distal end 1710 is angled with respect to the rest of the shaft 1702. By way of the example, the angled portion of the distal end 1710 can be angled from between about 20 and about 30 degrees with respect to the main axis of the shaft 1702. The angled portion can be from about 2 to about 3 centimeters in length. The tunneling instrument 1700 also has a lock fitting 1708, such as a LUER-LOKQR fitting, that can be used to secure the tunneling instrument 1700 to other pieces of equipment.

FIG. 38 is a cross-sectional view of the shaft 1702 of the tunneling instrument 1700 taken along line L-L′ of FIG. 37. The shaft 1702 includes a side wall 1714 and a lumen 1716. The lumen 1716 is large enough to allow passage of a 0.035 inch diameter (1 mm) guide wire. In some embodiments, the tunneling instrument 1700 is made of a metal. By way of example, the tunneling instrument 1700 can be made of stainless steel. In some embodiments, the tunneling instrument 1700 is made from an alloy such as Nitinol.

FIG. 39 shows an alternative embodiment of a tunneling instrument 1800. The tunneling instrument 1800 has a shaft 1802 and a distal end 1810. The distal end 1810 has a blunt tip to allow for the atraumatic dissection of the extraluminal tract. In this embodiment, the distal end 1810 is not angled with respect to the major axis of the shaft 1802. The tunneling instrument 1800 also has a lock fitting 1808, such as a LUER-LOK®, that can be used to secure the tunneling instrument 1800 to other pieces of equipment.

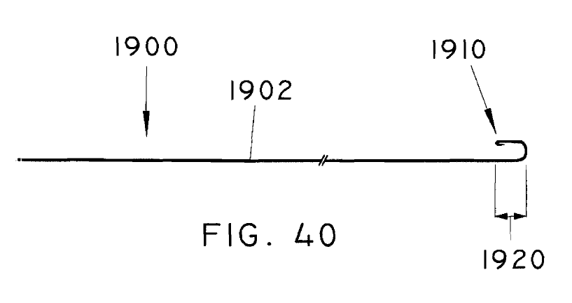

FIG. 40 shows an embodiment of a precurved guide wire 1900. The guide wire 1900 has a shaft 1902 and a distal end 1910. The distal end 1910 includes a curve with respect to the main axis of the shaft 1902. In some embodiments, this curve is about 180 degrees. The curved segment can have a length 1920 of approximately 10 centimeters. The precurved guide wire 1900 can be used for the atraumatic dissection of an extraluminal tract for placement of a graft. In some embodiments, the guide wire 1900 is made from an alloy, such as Nitinol. The guide wire 1900 can have a platinum floppy tip to facilitate visualization of the guide wire within the vascular lumen.

### Shape Memory Metals

Some grafts or instruments (or components thereof) described herein can include shape memory metals. Shape memory metals (or shape memory alloys) are a group of metals that have the ability to return to a previously defined shape or size when heated above a transition temperature. Typically, these materials can be plastically deformed at some relatively low temperature and upon exposure to a higher temperature will return to their shape prior to the deformation. Most of the transformation occurs over a relatively narrow temperature range, although the beginning and end of the transformation during cooling and heating extends over a much larger temperature range. The martensitic transformation that occurs in the shape memory alloy yields a thermoelastic martensite and this property is what defines shape memory alloys.

Shape memory metals include alloys of nickel-titanium (such as Nitinol); cobalt-based alloys (such as Elgeloy); nickel-based superalloys (such as Hastelloy or Incoloy) and different grades of stainless steel. Of these alloys, Nitinol has proven to have unique properties that makes it suitable for medical applications, including the fact that is extremely corrosion resistant, has excellent biocompatibility, can be fabricated into very small sizes, it is super-elastic and provides proportional control properties that allows to use only a part of the shape recovery as a way of restricting the opening of a conduit and therefore, to limit the flow through the structure.

While the present invention has been described with reference to several particular implementations, those skilled in the art will recognize that many changes may be made hereto without departing from the spirit and scope of the present invention. By way of example, while the grafts of the present invention were exemplified as femoropopliteal grafts, it will be appreciated that they can also be used as hemodialysis arteriovenous shunts, amongst other applications.

1-38. (canceled)

1. A method for percutaneous insertion of a femoropopliteal bypass graft comprising:
forming a first aperture in a first wall of a first artery;
forming a second aperture in a second wall of the first artery;
forming an extraluminal tract between the second aperture and a second artery;
forming a third aperture in the second artery, the extraluminal tract providing fluid communication between the second aperture and the third aperture;
passing the femoropopliteal bypass graft through the first and second apertures, through the extraluminal tract, and into the third aperture so that a first end of the femoropopliteal bypass graft is disposed within the first artery and a second end of the femoropopliteal bypass graft is disposed with the second artery.

2. The method of claim 1, the second wall of the first artery being opposite the first wall of the first artery.

3. The method of claim 1, further comprising occluding blood flow through the first and second arteries.

4. The method of claim 3, wherein occluding blood flow through the first and second arteries occurs before forming a third aperture the second artery.

5. The method of claim 1, wherein the first artery is a popliteal artery.

6. The method of claim 1, wherein the second artery is a superficial femoral artery.

7. The method of claim 1, wherein the second artery is a common femoral artery.

8. A femoropopliteal bypass graft comprising:
a first layer forming a cylinder having a first end and a second end, the first layer defining a lumen, the first layer comprising a biocompatible polymer;
a second layer forming a cylinder having a first end and a second end, the second layer disposed over the first layer; and
a third layer forming a cylinder having a first end and a second end;
the distance between the first end and the second end of the third layer being at least one centimeter less than the distance between the first end and the second end of the second layer.

9. The femoropopliteal bypass graft of claim 8, the second layer comprising a metal.

10. The femoropopliteal bypass graft of claim 8, the second layer comprising a shape-memory metal.

11. The femoropopliteal bypass graft of claim 8, the second layer comprising Nitinol.

12. The femoropopliteal bypass graft of claim 8, the second layer comprising a shape-memory metal woven together with a biocompatible polymer.

13. The femoropopliteal bypass graft of claim 8, the first layer comprising expanded polytetrafluoroethylene.

14. The femoropopliteal bypass graft of claim 8, the third layer comprising expanded polytetrafluoroethylene.

15. The femoropopliteal bypass graft of claim 8, the distance between the first end and the second end of the third layer being at least two centimeters less than the distance between the first end and the second end of the second layer.

16. The femoropopliteal bypass graft of claim 8, the distance between the first end and the second end of the third layer being at least one centimeter less than the distance between the first end and the second end of the first layer.

17. The femoropopliteal bypass graft of claim 8, the lumen defined by the first layer having a smaller diameter at the first end than at the second end.

18. A femoropopliteal bypass graft comprising:
a first layer forming a cylinder having a first end and a second end, the first layer defining a lumen, the first layer comprising a biocompatible polymer; and
a second layer forming a cylinder having a first end and a second end, the second layer disposed over the first layer, the second layer comprising a metal mesh;
wherein the biocompatible polymer comprises expanded polytetrafluoroethylene.

19. The femoropopliteal bypass graft of claim 18, the distance between the first end and the second end of the first layer being at least one centimeter less than the distance between the first end and the second end of the second layer.

20. The femoropopliteal bypass graft of claim 18, the second layer comprising a metal mesh mixed with a biocompatible polymer.
